(19) United States
(12) Patent Application Publication
(10) Pub. No.: US 2013/0166001 A1
(43) Pub. Date: Jun. 27, 2013

(54) CONTINUOUS-WAVE OPTICAL STIMULATION OF NERVE TISSUE

(71) Applicants: Nathaniel Michael Fried, Concord, NC (US); Serhat Tozburun, Charlotte, NC (US)

(72) Inventors: Nathaniel Michael Fried, Concord, NC (US); Serhat Tozburun, Charlotte, NC (US)

(73) Assignee: UNIVERSITY OF NORTH CAROLINA AT CHARLOTTE, Charlotte, NC (US)

(21) Appl. No.: 13/530,465

(22) Filed: Jun. 22, 2012

Related U.S. Application Data
(60) Provisional application No. 61/500,328, filed on Jun. 23, 2011.

(51) Int. Cl. A61N 5/06

(52) U.S. Cl. CPC A61N 5/0622
USPC 607/89

(57) ABSTRACT

An optical nerve stimulation system for optically stimulating and locating a nerve, includes: a laser radiation source positioned adjacent to the nerve and configured to deliver laser radiation to the nerve through one or more optical fibers, thereby heating it; wherein the laser radiation source is operated in a continuous-wave mode; and a temperature sensor positioned adjacent to the nerve and a feedback loop configured to control the laser radiation source such that a predetermined temperature is maintained. The laser radiation source is one or more of an infrared laser and a near-infrared laser. Optionally, the laser radiation has a wavelength of between 1400 and 1900 nm. The one or more optical fibers are one or more single-mode fibers. The ONS probe includes one or more SMFs with chemically-etched tips and lenses for providing collimation and/or beam shaping to provide a flat-top spatial beam profile.

### CROSS-REFERENCE TO RELATED APPLICATION(S)

The present patent application/patent claims the benefit of priority of co-pending U.S. Provisional Patent Application No. 61/500,328, filed on Jun. 23, 2011, and entitled “CONTINUOUS-WAVE OPTICAL STIMULATION OF NERVE TISSUE,” the contents of which are incorporated in full by reference herein.

### STATEMENT REGARDING FEDERALLY SPONSORED RESEARCH OR DEVELOPMENT

The present invention was made, in part, with the support of the U.S. Government pursuant to Department of Defense Award No. PC073709 and Department of Energy Award No. DE-FG02-06CH11460. Accordingly, the U.S. Government may have certain rights in the present invention.

### FIELD OF THE INVENTION

The present invention relates generally to systems and methods for the continuous-wave (CW) optical stimulation of nerve tissue. More specifically, the present invention relates to systems and methods for the CW infrared (IR) or near-infrared (near-IR) optical stimulation of the cavernous nerves (CNs) of the prostate gland or the like.

### BACKGROUND OF THE INVENTION

The identification of the CNs during prostate cancer surgery and the like is critical in preserving a man's ability to have spontaneous erections following surgery. Because of the close proximity of the CNs to the prostate surface, beneath a thin fascia layer, they are at risk of injury during the dissection and removal of a cancerous prostate gland. Their microscopic nature and location beneath this thin fascia layer make it difficult to predict the position and path of the CNs from one patient to another. These observations may, in part, explain the wide variability in sexual potency rates (9-86%) following prostate cancer surgery. Recent anatomic studies also suggest that the CNs may have more extensive branching along the prostate surface than originally thought, and that our current knowledge of the position and path of these nerves may be limited. Thus, the development of improved diagnostic methods for the identification of the CNs during prostate cancer surgery and the like would prove valuable for preserving the nerves and improving post-operative sexual function and patient quality of life.

Conventional electrical nerve stimulation (ENS) of the CNs is currently used in the laboratory to measure erectile response. Intra-operative electrical nerve mapping devices have also been tested as surgical diagnostic tools to assist in the preservation of the CNs during nerve-sparing prostate cancer surgery and the like. However, these electrical nerve mapping devices have proven inconsistent and unreliable in identifying the CNs and evaluating nerve function. Lack of specificity, high false-positive responses, and the influence of multiple conflicting factors in recording electrical responses during surgery have all been cited as significant limitations of electrical nerve mapping techniques.

Conventional ENS, in general, has several significant limitations. First, ENS is limited by the need for physical contact between the electrode and the tissue, which may result in nerve damage. Second, the spatial precision of ENS is limited by the electrode's size and electrical current spreading in the tissue. Third, ENS produces electrical artifacts that may interfere with measurement.

Recently, optical nerve stimulation (ONS) using pulsed IR laser radiation at relatively low pulse repetition rates (2-13 Hz, for example), presumably to avoid thermal build-up and damage to the nerves during long-term stimulation applications, and using multi-mode fiber (MMF) has been developed as a potential alternative to ENS. ONS, in general, offers several advantages, including: (1) a non-contact method of stimulation, (2) improved spatial selectivity, and (3) the elimination of stimulation artifacts. Conventional ONS methods, however, require large, powerful, and expensive laser delivery systems, making them impractical for efficient adoption by clinicians.

Thus, what is still needed in the art are improved ONS systems and methods that remedy these and other deficiencies of conventional ENS and ONS systems and methods, thereby resulting in better patient care and outcomes.

### BRIEF SUMMARY OF THE INVENTION

In various exemplary embodiments, the present invention provides open, minimally-invasive, laparoscopic, and/or robotically-assisted, intra-operative diagnostic systems and methods for the CW IR or near-IR optical stimulation of nerves, such as the CNs of the prostate gland or the like. The wayelengths utilized typically range from about 1400-1900 nm, in a preferred embodiment of the present invention. Single-mode fiber (SMF) is used, which, along with the use of a CW signal, allows smaller and less expensive, but higher quality, laser diodes to be used (30-100 mW instead of 5-10 W, for example), providing a compact, convenient, and inexpensive plug-and-play system that may be easily assembled and/or handled and used by a clinician or the like. Advantageously, the use of a CW signal allows for the deposition of more energy faster, in a manner that is less likely to damage the nerves. Optionally, the present invention utilizes a collimated beam and beam shaping via an environmentally-sealed probe incorporating a pen housing, chemically-etched fiber optic tip, and/or integrated lens or lens system, thereby allowing effective and efficient subsurface stimulation through Overlying tissue, such as thin fascia layers and the like. Finally, temperature-controlled ONS may be achieved using an IR sensor or the like and a closed-loop feedback system, such that nerve damage may be completely avoided. The effective stimulation and location of a nerve or nerve bundle, even a subsurface nerve or nerve bundle, in 3-30 sec of exposure may be achieved, for example. Longer term use is possible with the use of the closed-loop feedback system. The wavelengths utilized by the systems and methods of the present invention allow for deeper penetration, with less power, and less heat, and the beam collimation/beam shaping provides a broader range of working distances between the probe tip and the nerve surface (10-30 mm, for example) for effective operation than systems and methods that use more rapidly diverging non-collimated conical beams or the like. A mechanical shutter on the laser may be utilized to ensure that a probed nerve fires (typically at about 43 degrees C.), but that the probed nerve is not damaged (typically at about 46-48 degrees C.). The systems and methods of the present invention, being dependent upon temperature, are generally easier to calibrate than conventional pulsed laser systems and methods, being dependent upon numerous laser parameters, including: pulse energy, pulse duration, pulse rate, spot diameter, and irradiation time. Further, auto-scan applications may be developed and utilized. In general, the compact ONS probe of the present invention represents a single, user-friendly open or laparoscopic unit that incorporates the laser radiation delivery optics and fiber, the optical shutter, the IR detector for temperature measurement, and control and feedback software. Temperature accuracy should be within about 1 degree C. and response time should be about 0.1 s, with a nerve temperature of about 45 degrees C. being reached in about 2 s and maintained, for a total laser irradiation time of about 10 s, for example.

In one exemplary embodiment, the present invention provides an optical nerve stimulation (ONS) system for optically stimulating and locating a nerve associated with an anatomical structure, including: a laser radiation source positioned adjacent to the nerve and configured to deliver laser radiation to the nerve through one or more optical fibers, thereby heating it; wherein the laser radiation source is operated in a continuous-wave (CW) mode; and a temperature sensor positioned adjacent to the nerve and a feedback loop configured to control the laser radiation source such that a predetermined temperature is maintained at the nerve. The laser radiation source is one or more of an infrared (IR) laser radiation source and a near-infrared (near-IR) laser radiation source. The laser radiation has a wavelength of between about 1400 and about 1900 nm. The one or more optical fibers are one or more single-mode fibers (SMFs). The laser radiation is collimated by one or more lenses. The laser radiation is beam shaped by one or more of a chemically-etched fiber optic tip and a lens. The beam shape of the laser radiation includes a flat top. The ONS system also includes an optical shutter for selectively and rapidly turning the laser radiation on/off at the direction of the feedback loop. The temperature sensor is an IR radiometer. The nerve is a cavernous nerve (CN) of a prostate gland disposed beneath a thin fascia layer.

In another exemplary embodiment, the present invention provides an optical nerve stimulation (ONS) method for optically stimulating and locating a nerve associated with an anatomical structure, including: providing a laser radiation source positioned adjacent to the nerve and configured to deliver laser radiation to the nerve through one or more optical fibers, thereby heating it; wherein the laser radiation source is operated in a continuous-wave (CW) mode; and providing a temperature sensor positioned adjacent to the nerve and a feedback loop configured to control the laser radiation source such that a predetermined temperature is maintained at the nerve. The laser radiation source is one or more of an infrared (IR) laser radiation source and a near-infrared (near-IR) laser radiation source. The laser radiation has a wavelength of between about 1400 and about 1900 nm. The one or more optical fibers are one or more single-mode fibers (SMFs). The laser radiation is collimated by one or more lenses. The laser radiation is beam shaped by one or more of a chemically-etched fiber optic tip and a lens. The beam shape of the laser radiation includes a flat top. The ONS method also includes providing an optical shutter for selectively and rapidly turning the laser radiation on/off at the direction of the feedback loop. The temperature sensor is an IR radiometer. The nerve is a cavernous nerve (CN) of a prostate gland disposed beneath a thin fascia layer.

In a further exemplary embodiment, the present invention provides an optical nerve stimulation (ONS) probe for optically stimulating and locating a nerve associated with an anatomical structure, including: a housing coupled to one or more single-mode fibers (SMEs); one or more of an infrared (IR) laser radiation source and a near-infrared (near-IR) laser radiation source coupled to the one or more SMFs and configured to deliver laser radiation to the nerve in operation through the one or more SMFs, thereby heating the nerve; wherein the laser radiation source is operated in a continuous-wave (CW) mode; and one or more lenses and/or other means disposed within the housing for collimating and beam shaping the laser radiation. The ONS probe also includes an IR radiometer and a feedback loop coupled to the one or more of the IR laser radiation source and the near-IR laser radiation source, wherein the IR radiometer and the feedback loop are configured to control the one or more of the IR laser radiation source and the near-IR laser radiation source such that a predetermined temperature is maintained at the nerve. The nerve is a cavernous nerve (CN) of a prostate gland disposed beneath a thin fascia layer.

### BRIEF DESCRIPTION OF THE DRAWINGS

The present invention is illustrated and described herein with reference to the various drawings, in which like reference numbers are used to denote like system components and/or method steps, as appropriate, and in which:

FIG. 1 is a schematic diagram illustrating one exemplary embodiment of the CW ONS system of the present invention;

FIG. 2 is a schematic diagram illustrating one exemplary embodiment of the closed-loop feedback system of the present invention;

FIG. 3 is a schematic diagram illustrating another exemplary embodiment of the real-time temperature-controlled all-SMF ONS system 40 of the present invention;

FIG. 4 is a plot providing a representative range of intracavernous pressure (ICP) responses with repeated sub-surface ONSs in a single test rat; and

FIG. 5 is a plot providing another representative CW optical stimulation of a rat CN.

### DETAILED DESCRIPTION OF THE INVENTION

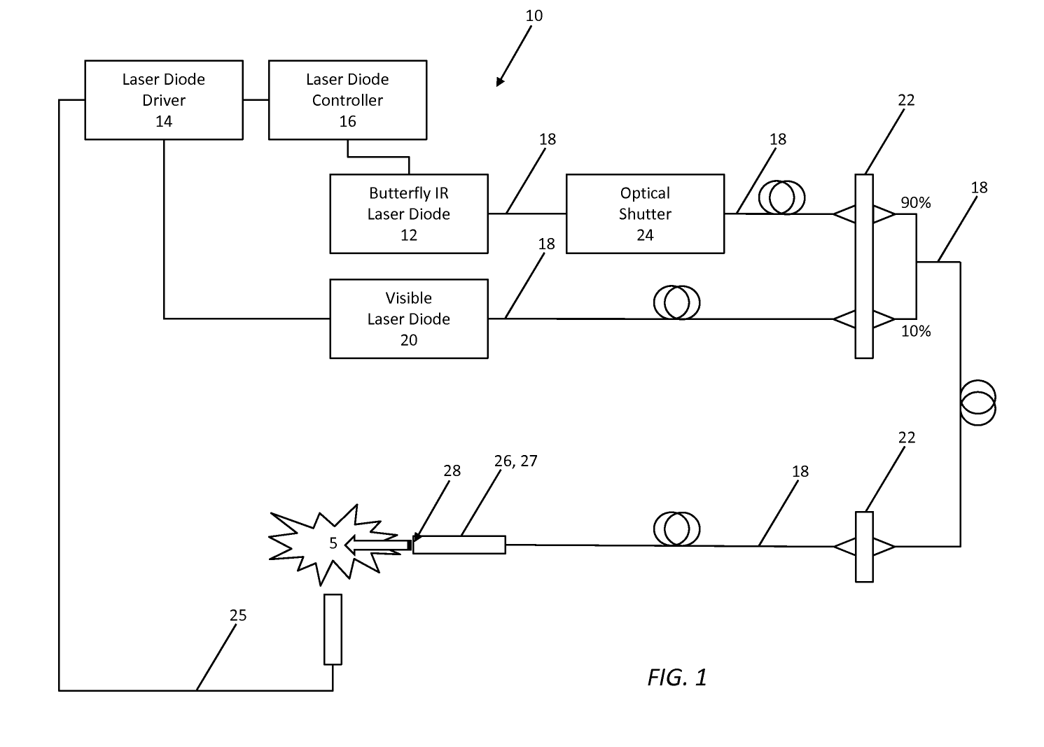

Referring to FIG. 1, in one exemplary embodiment of the present invention, the CW ONS system 10 includes an IR or near-IR butterfly laser diode assembly 12 or the like that is coupled to a laser diode driver 14 and a laser diode controller 16. Collectively, the laser diode assembly 12, laser diode driver 14, and laser diode controller 16 are operable for delivering CW laser radiation with a wavelength of about 500-3000 nm, preferably with a wavelength of about 1000-2500 nm, and more preferably with a wavelength of about 1000-2000 nm, and most preferably with a wavelength of about 1400-1900 nm, in the IR or near-IR ranges. The laser diode assembly 12 is coupled to one or more lengths of SMF 18 that is/are used to deliver the laser radiation to the surgical site 5, optionally through one or more optical couplers 22 or the like. The SMF 18 ultimately provides a spot diameter of 0.1-5 mm, for example, and preferably a spot diameter of 0.5-2 mm. A visible aiming laser diode assembly 20, operable for delivering laser radiation with a wavelength of about 400-700 nm, for example, may also be tied into the SMF 18, optionally in a parallel arrangement using a 90%-10% fiber coupler 22 or the like, such that a visible aiming reference is provided to the surgical site 5 through the SMF 18, as well as the primary laser radiation. An optical shutter 24 or the like (such as an optical attenuator) is disposed between the laser diode assembly 12 and the surgical site 5, such that laser radiation delivered to the surgical site 5 can be selectively blocked (wholly or partially) during normal operation, in the event of an emergency, and/or in the event that a closed-loop feedback system 25 or the like, described in greater detail herein below, determines that the surgical site 5 has been heated to too great a degree, for example. During normal operation and/or in the event that the closed-loop feedback system 25 or the like determines that the surgical site 5 has been heated to too great a degree, the laser power may also be adjusted independent of the optical shutter 24 or the like using the laser diode controller 16. The SMF 18 terminates adjacent to the surgical site 5 in a chemically-etched tip, a ferrule 26, a pen housing 27 or the like, and/or one or more lenses 28. The pen housing 27 or the like allows the “probe” at the end of the SMF 18 to be handheld or robotically controlled during use. Alternatively, some or all of the SMF 18 is also disposed within a housing. The wavelengths utilized by the systems and methods of the present invention allow for deeper penetration, with less power, and less heat, and the beam collimation/beam shaping resulting from the use of the chemically-etched tip and/or one or more lenses 28 provides a broader range of working distances between the probe tip and the nerve surface for effective operation (10-30 mm, for example) than systems and methods that use “messier” non-collimated conical beams or the like. They also result in a more effective and stable heating of the nerves for stimulation.

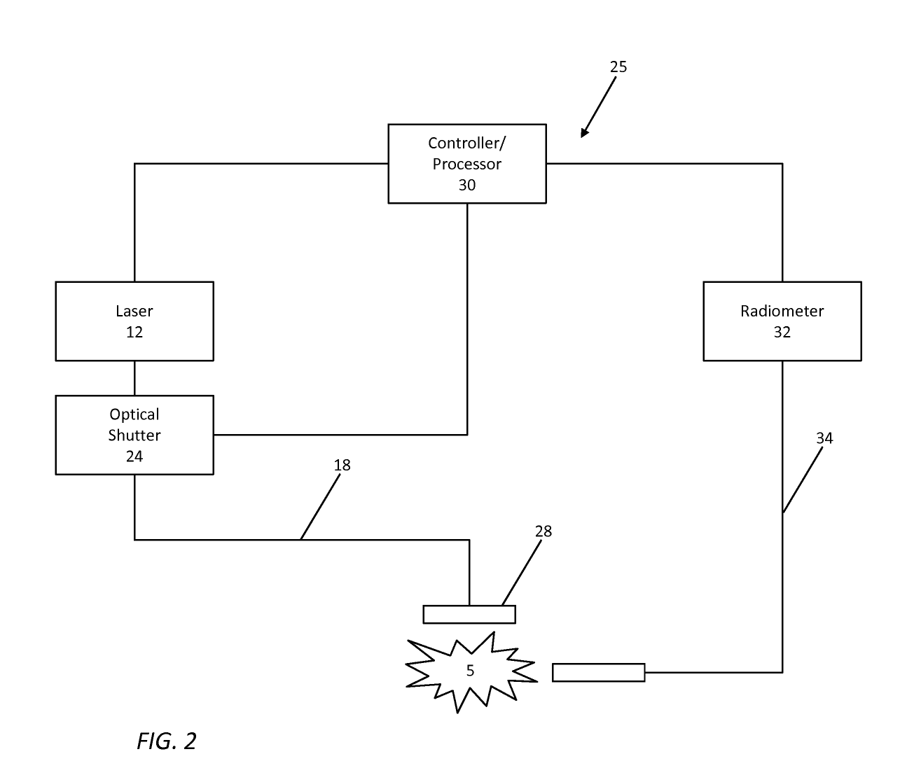

Referring to FIG. 2, in one exemplary embodiment of the present invention, the closed-loop feedback system 25 includes a controller/processor 30 that is coupled to the laser 12 and the shutter 24, as well as a radiometer 32 that is configured to determine the temperature present at the surgical site 5 with a high degree of accuracy. I f the temperature is too high, the controller 30 directs the laser 12 to reduce its power output or the shutter 24 to “shut off” the laser radiation that is delivered to the surgical site 5, such that nerve damage is prevented. If the temperature is too low, the controller 30 directs the laser 12 to increase its power output, such that nerve stimulation is assured. This is accomplished through the delivery SMF 18 and a sensor SMF or electrical lead 34. The radiometer 32 may be replaced with any other suitable temperature sensing system and is described in greater detail herein below.

Radiometry is based on the principle that any tissue with a temperature above absolute zero emits IR. According to the Stephan-Boltzmann law, the intensity, l₀, of thermal radiation emitted from a surface tissue area, A, is given by l₀=AεσT⁴, where ε is the emissivity, σ is the Stephan-Boltzmann constant, 5.67×10⁻¹² Wm⁻² K⁻⁴, and T is the tissue temperature. The emissivity, ε, is determined by the tissue's surface properties. For soft tissue, ε is approximately 1, due to its large water content. In addition, thermal radiation energy is homogenous in all directions. Incoherent radiation sources, like emitters and scatterers, produce radiation whose radiance is completely independent of viewing angle, which can be described by a Lambertian (diffuse) type of emission, given by l=l₀ cos θ. This is Lambert's cosine law, where θ is the viewing angle and l is the intensity of a ray leaving in a direction cosine of θ to the surface. From Wien's law, tissues at different body temperatures emit radiation at different peak wavelengths: Tλₚₑₐₖ=w, where λₚₑₐₖ is the wavelength of peak blackbody emission at an absolute temperature of T and w is called Wien's displacement constant, equal to 2898 μm K. For the temperature range of 305-323 K (32-50 degrees C.), typical for ONS studies, thermal radiation is emitted primarily in the spectral range of 8.9-9.5 μm.

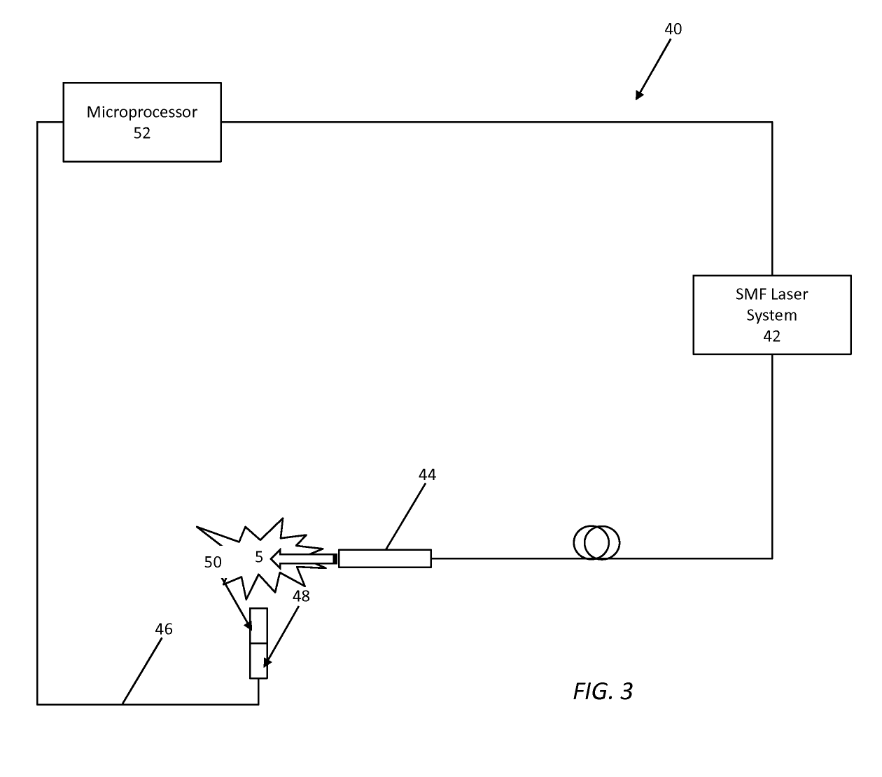

Referring to FIG. 3, in another exemplary embodiment of the present invention, the real-time temperature-controlled all-SMF ONS system 40 includes an all-SMF system 42, a laser probe 44, and a radiometer temperature feedback control system 46. The radiometer system 46 includes a thermopile digital sensor module 48, a plano-convex IR lens 50 made of zinc selenide (ZnSe) with transmission in the spectral range of 600 nm-16 μm, and a microprocessor 52. The thermopile sensor 48 has an absorbing area of 0.37 mm², and it is sensitive to the 5.5-13.5 μm spectral range and generates a pulse-width modulation (PWM) signal with 10 bit resolution that is proportional to the temperature. The response time of the detector is 5 ms with a refresh rate of between 100 and 250 ms. The mid-IR radiation emitted from the heated nerve surface is collected and transmitted to the IR sensor 48 generating a signal by the use of the focusing lens 50. The output signal from the IR sensor 48 is processed by the microprocessor 52 for data acquisition and control. The actual control of the laser power over the nerve surface is accomplished by providing rapid on/off switching with the optical shutter 24 (FIGS. 1 and 2).

The experimental setup that is used to calibrate the radiometer 32 (FIG. 2) includes a black anodized aluminum plate with an emissivity of about 0.9, used as a blackbody object. Control of the surface temperature of the blackbody object is achieved using a heating plate. A thermocouple attached to the surface of the blackbody object is used to measure the surface temperature with a resolution of 1 degree C. The radiometer 32 is placed at a fixed distance (2.5 cm, for example) above the blackbody object surface and the IR signal is recorded at a range of temperatures (between 30 and 50 degrees C. for example).

The feasibility of the ONS system 10 (FIG. 1) and 40 (FIG. 3) of the present invention was studied in rats with a fascia layer placed over the CNs, as in humans. Two near-IR lasers 12 (FIG. 1) with wavelengths of 1455 and 1550 nm, respectively, were operated in CW mode for stimulation of the CNs in eight rats, in vivo. Successful ONS was confirmed by an intracavernous pressure (ICP) response in the rat penises at 1455 nm through fascia with a thickness up to 110 μm and at 1550 nm through fascia with a thickness up to 450 μm. Higher incident laser power was required to produce an ICP response as fascia thickness was increased. Also, weaker and slower ICP responses were observed as fascia thickness was increased.

All studies were performed under an animal protocol approved by the Animal Care and Use Committee at Johns Hopkins Hospital. Eight Sprague Dawley rats (400-600 g) were anesthetized by intraperitoneal injection with 50 mg/kg sodium pentobarbital. The rats were secured in the supine position and prepped for surgery. The CN arising from the ipsilateral major pelvic ganglion situated dorsolateral to the prostate was exposed via a midline suprapubic incision and anterior pelvic dissection. To assess ICP, the shaft of the penis was denuded of skin and the left crural region was cannulated with a 23 G needle connected via polyethylene tubing to a pressure transducer (Harvard Apparatus, Holliston, Mass.). An increase in ICP after optical stimulation of the CN was detected by a data acquisition system (D1-190, Dataq Instruments, Akron, Ohio). ICP (mmHg) response as a function of time(s) was plotted using MATLAB 6.0 software (Mathworks, Natick, Mass.) with a data acquisition rate of 8 Hz and an accuracy to within 0.1 mmHg. This method is based on a standard technique for measuring ICP. As mentioned, the ONS experiments were performed under an approved animal protocol, and at the completion of the study, the rats were euthanized by intracardiac injection of potassium chloride while under anesthesia, as is consistent with the recommendations of the Panel of Euthanasia of the American Veterinary Medical Association.

A compact digital microscope with variable (20×-90×) magnification (AD413TL, Dino-lite, Torrance, Calif.) was focused onto the prostate surface to assist in alignment of the red (660 nm) diode laser aiming beam with the CN. A thermal camera (A20M, Flir Systems, Boston, Mass.) provided temperature-mapping of the CN during ONS to provide temperature feedback during the procedure. The following sequence of steps was used in the experiments. First, the visible aiming beam was used to align the 1.0 mm-diameter IR laser beam with the exposed CN. Second, ONS was performed to determine the threshold laser power for stimulation by slowly escalating the laser power in small (5 mW) increments and then repeating stimulation. Third, a thin layer of fascia from the rat testicle was dissected and then placed directly over the exposed CN. Fourth, an optical coherence tomography system (Niris, lmalux, Cleveland, Ohio) with compact 8 Fr (2.7 mm-OD) endoscopic probe placed in gentle contact with the tissue was used to non-invasively image and measure the thickness of the fascia layer with an axial resolution of approximately 10 μm. Fifth, subsurface ONS was performed again by slowly escalating the laser power in small (5 mW) increments until the threshold for stimulation was reached, as confirmed by an ICP response in the rat penis. During these steps, the CN and fascia surfaces were periodically rinsed with saline to prevent desiccation during the experiments.

Two pigtailed, single-mode, near-IR diode lasers were operated in CW mode for this study. The first laser (QFLD-1450, Qphotonics, Ann Arbor, Mich.) emitted up to 150 mW of power at a wavelength of 1455 nm. Laser radiation at 1455 nm has an optical penetration depth (OPD) of about 350 μm in water (the dominant soft tissue absorber in the near-IR), which closely matches the rat CN diameter (200-400 μm) for uniform irradiation of the nerve. This 1455 nm diode laser also provides a similar OPD in water to that of the 1870 nm Thulium fiber laser used in previous studies. The 1455 nm laser was used for two purposes: (1) to demonstrate a robust ONS response prior to placement of the fascia layer over the CN and (2) then to determine the limits of this wavelength in ONS as a function of fascia thickness. The second laser (Brightlase-7115, Laser Operations LLC, Sylmar, Calif.) emitted up to 500 mW of power at a wavelength of 1550 nm. Laser radiation at 1550 nm has an OPD of about 930 μm in water. The purpose of using this more deeply-penetrating wavelength was to determine the limits of sub-surface ONS of the CN when a thicker layer of fascia was present. For both lasers, the experimental setup was similar: a red diode laser aiming beam (QFLD-660, QPhotonics) with a power of 20 mW and a wavelength of 660 nm was coupled into an SMF. An SMF coupler (1×2 SM Dual Window Coupler, FIS, Oriskany, N.Y.) with a 90/10 coupling ratio combined both visible and IR laser beams into a single SMF. The fiber coupler output was connected to a custom-built 10 Fr (3.4 mm-OD) laparoscopic probe consisting of 9 μm-core/125 μm-cladding SMF (SMF-28, Corning, N.Y.) with an aspheric lens (354430C, 2 mm-OD, 5 mm-focal length, Thorlabs, Newton, N.J.) attached to the distal tip for beam collimation. The custom 3.4 mm-OD laparoscopic probe provided a collimated 1.0 mm-diameter laser spot on the surface of the tissue at a working distance of 20 mm. An in-line optical shutter (SH-200-1480-9/125, Oz Optics, Ottawa, Canada) in the IR SMF arm provided rapid on/off switching of the laser beam during ONS. A laser power meter (FPM 1000, Coherent, Santa Clara, Calif.) and detector (PM10, Coherent) were used to measure the fiber probe output power incident on the tissue surface.

All ONS experiments were performed with an irradiation time of 15 s and a laser spot diameter of 1 mm. The incident laser power was increased in 5 mW increments from 30-90 mW until the threshold incident power for successful ONS was observed. A minimum of five stimulations were performed at each laser power level. The total energy delivered to the tissue ranged from 0.5-1.35 J. Fascia layers ranged from a thickness of 80-620 μm. For thin fascia layers (80-110 μm), only the 1455 nm laser was used for successful ONS. When the 1455 nm laser failed to produce an ICP response in thicker fascia layers, a 1550 nm laser was then substituted in the experiments. A successful ICP response was judged by the ability to clearly differentiate the peak ICP during ONS from the normal baseline ICP (10-15 mmHg) observed before and after ONS.

Successful optical stimulation of the CNs was achieved through a fascia layer with a thickness up to 450 μm. However, there was a significant decrease in the magnitude and speed of ICP response between optical stimulation of the CN on the surface versus sub-surface stimulation with a layer of fascia present, overlying the CN. The delay in stimulation between surface and sub-surface stimulation was approximately 3 s, with the initiation of an ICP response occurring about 9 s and 12 s after the start of laser irradiation, respectively. The difference in magnitude and shape of the ICP responses both between surface and sub-surface stimulation studies, and within each study as well, fluctuates and is dependent on anatomical and physiological parameters (e.g. nerve dimensions and blood pressure).

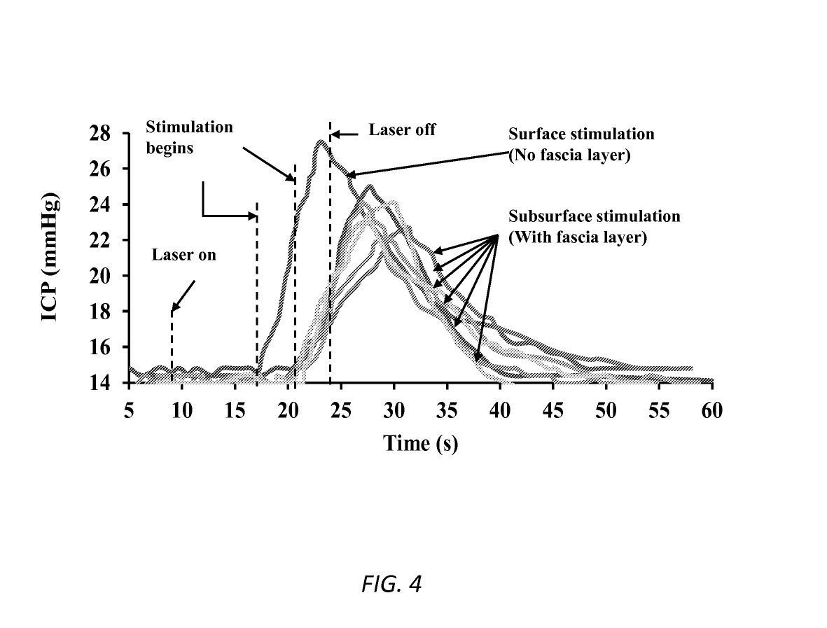

FIG. 4 provides a representative range of ICP responses with repeated sub-surface stimulations in a single rat. For the purposes of developing ONS as a potential intraoperative diagnostic method, an ICP response significantly above the baseline would be sufficient for positive identification of the CN during prostate surgery. In this study, the signal-to-noise ratio (SNR) was approximately 3:2, with baseline and peak ICP responses measuring approximately 10-15 mmHg and 20-25 mmHg, respectively. As the thickness of the fascia was increased from 80-620 μm, the threshold laser power for stimulation also increased (see Table 1 below). Successful ONS was observed at 1455 nm through a thin fascia layer measuring up to 110 μm thick. However, when the fascia layer was increased to a thickness of 240 μm, no ICP response was measured with the 1455 nm laser, and thermal damage to the fascia was observed at a power of 60 mW, corresponding to a peak temperature of about 48 degrees C. on the fascia surface. As an approximate first analysis, one can consider whether the combined fascia and nerve thickness was less than or greater than the OPD for the laser wavelength used when considering the success or failure of an experiment. The OPD at 1455 nm is about 350 μm, so when the thickness of the CN (about 200 μm) is also added to the fascia thickness, the CN depth is beyond the OPD for 1455 nm. Therefore, the failure to stimulate the CN with the 1455 nm laser and a fascia layer of 240 μm may be due to the combined fascia and nerve thickness being greater than the OPD at 1455 nm..

TABLE 1
Sub-surface ONS Threshold Parameters for the Rat CN as a Function of Laser Wavelength and Fascia Layer Thickness

| Parameter | Fascia Thickness (μm) | | | | | |
|---|---|---|---|---|---|---|
| (1455 nm/1550 nm) | 80-90 | 100-110 | 230-240 | 300-320 | 420-450 | 600-620 |
| power to stimulate | 33/NA | 45/NA | No Stim./45 | No Stim./60 | No Stim./80 | No Stim./No Stim. |
| (mW): | | | | | | |
| Threshold ICP | 13.8 ± 0.3/NA | 14.4 ± 0.5/NA | NA/14.8 ± 0.3 | NA/14.1 ± 0.3 | NA/13.1 ± 0.5 | NA/NA |
| response time (s): | | | | | | |
| Total energy in | 0.46/NA | 0.65/NA | NA/0.67 | NA/0.85 | NA/1.05 | NA/NA |
| stimulate (J): | | | | | | |
| Total fluence to | 58.6/NA | 82.8/NA | NA/85.4 | NA/108.3 | NA/133.8 | NA/NA |
| stimulate (J/cm²): | | | | | | |
| Temperature on | 41.1 ± 0./NA | 41.5 ± 0.5/NA | NA/40.2 ± 0.3 | NA/44.8 ± 0.9 | NA/45.0 ± 0.6 | NA/NA |
| fascia (° C.): | | | | | | |

NA = not applicable
ICP = intracavernous pressure

Again, in FIG. 4, ONS was first performed without a fascia buffer, as a control, before then placing the fascia layer over the CN for sub-surface stimulation. The ICP response without fascia occurred approximately 9 s after laser irradiation began. Although sub-surface ONS was reproducible, as shown by the six individual stimulations performed, the ICP response was significantly weaker and averaging approximately 3 s slower than ONS without the fascia layer. Tissue thickness=450 μm. Laser Parameters: Wavelength=1550 nm, Power=80 mW.

For thicker fascia layers (240-600 μm), the 1550 nm laser with an OPD of about 930 μm was substituted for the 1455 nm laser. Successful ONS was achieved in fascia layers up to 450 μm thick, but no ICP response was detected for a 600 μm fascia layer. A threshold laser power of 45 mW was necessary for a 240 μm fascia layer versus 80 mW for the 450 μm fascia layer. Although the 1550 nm laser was capable of output powers up to 500 mW, thermal damage to the fascia layer was observed when the laser power was increased to 90 mW for the 600 μm fascia layer, corresponding to a peak temperature of approximately 53 degrees C. on the fascia surface. The thickest fascia layer in which successful ONS was observed at 1550 nm, was 450 μm, which corresponded to a laser power of 80 mW at the threshold for stimulation and a temperature of about 45 degrees C. at the surface of the fascia.

In a prior example, seven Sprague Dawley rats (400-600 g) were anesthetized by intraperitoneal injection with 50 mg/kg sodium pentobarbital. The rats were secured in the supine position and prepped for surgery. The CN arising from the ipsilateral major pelvic ganglion situated dorsolateral to the prostate was exposed via a midline suprapubic incision and anterior pelvic dissection. To assess ICP, the shaft of the penis was denuded of skin and the left crural region was cannulated with a 23 G needle connected via polyethylene tubing to a pressure transducer (Harvard Apparatus, Holliston, Mass.). An increase in ICP after optical stimulation of the CN was detected by a data acquisition system (DI-190, Dataq Instruments, Akron, Ohio). The response parameters were analyzed with MATLAB software (Mathworks, Natick, Mass.). The ONS experiments were performed under an approved animal protocol, and at the completion of the study, the rats were euthanized by intracardiac injection of potassium chloride while under anesthesia, as is consistent with the recommendations of the Panel of Euthanasia of the American Veterinary Medical Association. Optical nerve stimulation was performed with a Thulium fiber laser (TLT-5, IPG Photonics, Oxford, Mass.) using a similar wavelength (λ=1870 nm) and pulse duration (5 ms) as previously reported. The laser radiation was coupled into a custom-built probe, consisting of a 200-μm-core, low-OH, silica optical fiber with an aspheric lens attached to the distal tip to deliver a collimated, flat-top, 1-mm-diameter laser spot at a working distance of 20 mm, as previously reported. Two laser parameters were varied for this study, the pulse rate (10-100 Hz, CW) and the radiant exposure (0.03-0.67 J/cm²). The laser pulse energy was escalated until the radiant exposure reached the threshold for ONS. The ICP response was then measured at or just above the stimulation threshold, for safe and reproducible stimulation while preventing thermal damage to the nerve. The temperature of the CN was also recorded with a thermal camera (A20M, Flir Systems, Boston, Mass.) during ONS in an effort to optimize the laser stimulation parameters and to gain further insight into the mechanism of ONS. For each laser data set, a minimum of five ONSs were performed. The data reported in Table 2 for temperature, temperature change, and ICP response time represents the average of five independent measurements+the standard deviation (SD).

Table 2 provides a comprehensive summary of the results for the preliminary study of CW versus pulsed ONS. The threshold (minimum) total energy, average power, radiant exposure, and temperature for successful ONS are reported, along with the ICP response time, for a fixed laser stimulation time of 15 s. The stimulation threshold was observed to be directly dependent on the total energy delivered to the nerve, regardless of the mode or pulse rate used. This total energy also directly corresponded to a temperature increase in the nerve above a threshold temperature of 42-45 degrees C., at which ONS was first observed. The threshold radiant exposure for successful nerve stimulation was not fixed as previously thought, but rather decreased significantly as the laser pulse rate increased, effectively approaching quasi-CW mode. Finally, the ICP response time continued to decrease as the pulse rate was increased, with CW irradiation providing the fastest ICP response time.

TABLE 2
Optical Nerve Stimulation Threshold for CW and Pulsed Laser Irradiation With 15 s Stimulation

| | Laser Pulse Repetition Rate | | | | | | |
|---|---|---|---|---|---|---|---|
| Parameter | 10 Hz | 20 Hz | 30 Hz | 40 Hz | 50 Hz | 100 Hz | CW |
| Total Energy (mJ): | 0.73 | 0.77 | 0.77 | 0.80 | 0.71 | 0.83 | 0.71 |
| Average Power (mW): | 48.4 | 51.3 | 51.2 | 53.6 | 47.2 | 55.5 | 47.3 |
| Radiant Exposure (J/cm²): | 0.51 | 0.27 | 0.18 | 0.14 | 0.10 | 0.06 | NA |
| Temperature (° C.): | 43.8 ± 1.3 | 42.3 ± 1.1 | 42.3 ± 0.2 | 41.5 ± 0.6 | 44.9 ± 1.2 | 44.7 ± 1.2 | 42.9 ± 0.3 |
| ICP Response Time (s): | 16.7 ± 1.9 | 14.8 ± 1.3 | 14.5 ± 1.1 | 14.0 ± 0.5 | 12.8 ± 1.3 | 10.9 ± 1.6 | 9.7 ± 0.8 |

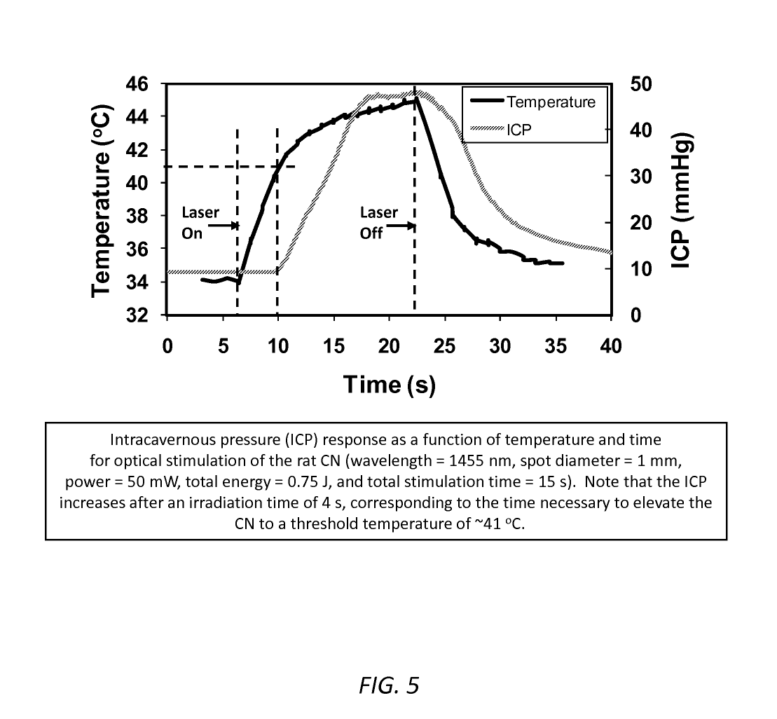

A representative example of CW optical stimulation of the rat CN is shown in FIG. 5. A strong response in the rat penis was observed with the ICP increasing from a baseline of 16 mmHg to a peak of 39 mmHg. This response occurred approximately 10 s after the laser was turned on, corresponding to the time necessary for the CN to heat up above a threshold temperature of approximately 43 degrees C. It is also part of the present invention to achieve even faster ICP responses if the laser power is increased further, so that the time necessary to raise the temperature from baseline to above 43 degrees C. decreases. For example, at higher laser powers, still safely below the threshold for thermal damage to the nerve, ICP response times as short as 4 s have been observed.

The mechanism of ONS is based on controlled heating of neural tissue. As an alternative system for ONS, a closed-loop, thermal-feedback-controlled laser delivery system (which is one object of the present invention) can be used. This system operates based on on/off laser radiation using a laser shutter. The hardware, controlled entirely by temperature feedback, is synchronized with the laser shutter, so that precise control of the surface temperature of the nerve is maintained during ONS.

In one exemplary embodiment of the present invention, two optical fibers are used. One optical fiber, the delivery fiber, is used for flexible delivery of the near-IR laser radiation to the nerve, producing a temperature rise in the nerve above the threshold temperature for ONS. A radiometer utilizing a second optical fiber, or sensing fiber, is used for maintaining the nerve temperature at a safe temperature above which ONS occurs (˜43° C.), but below the temperature at which thermal damage occurs to the nerve (˜47° C.). The sensing fiber provides a non-contact method for the continuous monitoring of the nerve temperature by measuring the IR thermal radiation emitted from the irradiated area of the nerve. A temperature range is specified in advance, such that if the nerve temperature drops below the stimulation threshold, then the laser is automatically turned on, and if the temperature rises too high, then the laser is automatically turned off.

In conclusion, the preservation of the CNs, which are responsible for sexual function, has proven challenging during prostate cancer surgery and the like, due to the close proximity of the CNs to the prostate surface. Their microscopic nature also makes it difficult to predict the location and path of these nerves from one patient to another. The implementation of new technologies which are capable of improved identification and detection of the CNs during surgery would aid in the preservation of the CNs and result in improved post-operative sexual function and patient quality-of-life.

ONS has recently been studied as an alternative to ENS. Although the exact mechanism of ONS using near-IR lasers is not yet known, previous studies have concluded that it is based primarily on a photothermal mechanism. Due to the IR wavelengths used and the long-pulse and CW mode of stimulation, other photochemical and photomechanical laser-tissue interactions have been ruled out as possible mechanisms for ONS. During our previous studies, we have also noticed a strong dependence of ONS on the temperature of the CN. The transformation of absorbed optical energy into sufficient heat deposited in the CN generates an increase in the nerve temperature above a threshold value of about 42-45 degrees C. for nerve activation and an increase in the ICP. A similar temperature range has previously been reported as necessary to open ion channels for nerve activation. The delivery of the optical energy in a CW mode results in faster deposition of the energy to the nerve, and therefore a faster ICP response as well.

This study demonstrates the feasibility of optical stimulation and identification of the rat CN when it lies beneath a thin layer of fascia, approximating the human anatomy during prostate cancer surgery. While the 1455 nm near-IR laser wavelength has proven successful, the OPD (about 350 μm) at this wavelength was not sufficient to successfully stimulate the CN when it was beneath a fascia layer thickness of 240 μm or greater. Therefore, it was necessary to switch to the 1550 nm laser wavelength, which has an OPD of 930 μm, for successful stimulation of the CN in fascia layers up to 450 μm thick. While the thickness of the fascia layer overlying the CNs in the human prostate is variable, previous detailed anatomic studies of the CNs in fresh human cadavers have revealed that the fascia layer varies from 100-300 μm in thickness. It should be noted that typically there is some shrinkage of tissue during histologic processing, and that these values may slightly underestimate the true thickness of the fascia layer. However, other studies using optical coherence tomography (OCT) to image the human cavernous nerves and prostate, in vivo, have shown comparable dimensions for the fascia layer. Therefore, demonstration of ONS of the rat CN through a fascia layer up to at least 450 μm in this study shows promise for future clinical translation of this ONS method into the human as well.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An optical nerve stimulation (ONS) system for optically stimulating and locating a nerve associated with an anatomical structure, comprising:
a laser radiation source positioned adjacent to the nerve in operation and configured to deliver laser radiation to the nerve in operation through one or more optical fibers, thereby heating it;
wherein the laser radiation source is operated in a continuous-wave (CW) mode.

2. The ONS system of claim 1, further comprising a temperature sensor positioned adjacent to the nerve in operation and a feedback loop configured to control the laser radiation source such that a predetermined temperature is maintained at the nerve.

3. The ONS system of claim 1, wherein the laser radiation source comprises one or more of an infrared (IR) laser radiation source and a near-infrared (near-IR) laser radiation source.

4. The ONS system of claim 3, wherein the laser radiation has a wavelength of between about 1400 and about 1900 nm.

5. The ONS system of claim 1, wherein the one or more optical fibers comprise one or more single-mode fibers (SMFs).

6. The ONS system of claim 1, wherein the laser radiation is collimated by one or more lenses.

7. The ONS system of claim 1, wherein the laser radiation is beam shaped by one or more of a chemically-etched fiber optic tip and a lens.

8. The ONS system of claim 7, wherein the beam shape of the laser radiation comprises a flat top.

9. The ONS system of claim 2, further comprising an optical shutter for selectively turning the laser radiation on/off at the direction of the feedback loop.

10. The ONS system of claim 2, wherein the temperature sensor comprises an IR radiometer.

11. The ONS system of claim 1, wherein the nerve comprises a cavernous nerve (CN) of a prostate gland disposed beneath a thin fascia layer.

12. An optical nerve stimulation (ONS) method for optically stimulating and locating a nerve associated with an anatomical structure, comprising:
providing a laser radiation source positioned adjacent to the nerve in operation and configured to deliver laser radiation to the nerve in operation through one or more optical fibers, thereby heating it;
wherein the laser radiation source is operated in a continuous-wave (CW) mode.

13. The ONS method of claim 12, further comprising providing a temperature sensor positioned adjacent to the nerve in operation and a feedback loop configured to control the laser radiation source such that a predetermined temperature is maintained at the nerve.

14. The ONS method of claim 12, wherein the laser radiation source comprises one or more of an infrared (IR) laser radiation source and a near-infrared (near-IR) laser radiation source.

15. The ONS method of claim 14, wherein the laser radiation has a wavelength of between about 1400 and about 1900 nm.

16. The ONS method of claim 12, wherein the one or more optical fibers comprise one or more single-mode fibers (SMFs).

17. The ONS method of claim 12, wherein the laser radiation is collimated by one or more lenses.

18. The ONS method of claim 12, wherein the laser radiation is beam shaped by one or more of a chemically-etched fiber optic tip and a lens.

19. The ONS method of claim 18, wherein the beam shape of the laser radiation comprises a flat top.

20. The ONS method of claim 13, further comprising providing an optical shutter for selectively turning the laser radiation on/off at the direction of the feedback loop.

21. The ONS method of claim 13, wherein the temperature sensor comprises an IR radiometer.

22. The ONS method of claim 12, wherein the nerve comprises a cavernous nerve (CN) of a prostate gland disposed beneath a thin fascia layer.

23. An optical nerve stimulation (ONS) probe for optically stimulating and locating a nerve associated with an anatomical structure, comprising:
a housing coupled to one or more single-mode fibers (SMFs);
one or more of an infrared (IR) laser radiation source and a near-infrared (near-IR) laser radiation source coupled to the one or more SMFs and configured to deliver laser radiation to the nerve in operation through the one or more SMFs, thereby heating the nerve;
wherein the laser radiation source is operated in a continuous-wave (CW) mode; and
one or more lenses and/or other means disposed within the housing for collimating and beam shaping the laser radiation.

24. The ONS probe of claim 23, further comprising an IR radiometer and a feedback loop coupled to the one or more of the IR laser radiation source and the near-IR laser radiation source, wherein the IR radiometer and the feedback loop are configured to control the one or more of the IR laser radiation source and the near-IR laser radiation source such that a predetermined temperature is maintained at the nerve.

25. The ONS probe of claim 23, wherein the nerve comprises a cavernous nerve (CN) of a prostate gland disposed beneath a thin fascia layer.
